(19) United States
(12) Patent Application Publication
(10) Pub. No.: US 2025/0000954 A1
(43) Pub. Date: Jan. 2, 2025

(54) SYSTEMS FOR ACCELERATING HYDROLYSIS OF IMPLANTABLE MEDICAL DEVICES

(71) Applicant: Boston Scientific Scimed, Inc., Maple Grove, MN (US)

(72) Inventors: Heather Cirka, Hudson, MA (US); Yen-Hao Hsu, Shrewsbury, MA (US); Marc Plourde, Worcester, MA (US); Joseph Thomas Delaney, JR., Minneapolis, MN (US); Cristian Parisi, Boston, MA (US)

(73) Assignee: Boston Scientific Scimed, Inc., Maple Grove, MN (US)

(21) Appl. No.: 18/754,776

(22) Filed: Jun. 26, 2024

Related U.S. Application Data
(60) Provisional application No. 63/510,413, filed on Jun. 27, 2023.

(51) Int. Cl. A61K 38/46; A61M 5/178; A61N 5/10

(52) U.S. Cl. CPC A61K 38/465; A61M 5/178; A61N 5/10; A61N 2005/1094

(57) ABSTRACT

Systems for hydrogel hydrolysis as well as methods for hydrogel hydrolysis are disclosed. An example system may include a syringe barrel having a needle connector disposed thereon. A hydrogel hydrolysis material may be disposed in the syringe barrel. The hydrogel hydrolysis material may be configured to be injected into a hydrogel formed in the body of a patient in order to accelerate the hydrolysis of the hydrogel.

### CROSS REFERENCE TO RELATED APPLICATIONS

This application claims the benefit of U.S. Provisional Patent Application Ser. No. 63/510,413 filed on Jun. 27, 2023, the disclosure of which is incorporated herein by reference.

### TECHNICAL FIELD

The present disclosure pertains to medical devices, and methods for manufacturing medical devices. More particularly, the present disclosure pertains to systems for accelerating hydrolysis of implantable medical devices.

### BACKGROUND

A wide variety of medical devices have been developed for medical use. These devices are manufactured by any one of a variety of different manufacturing methods and may be used according to any one of a variety of methods. Of the known medical devices and methods, each has certain advantages and disadvantages. There is an ongoing need to provide alternative medical devices as well as alternative methods for manufacturing and using medical devices.

### BRIEF SUMMARY

This disclosure provides design, material, manufacturing method, and use alternatives for medical devices. A system for hydrogel hydrolysis is disclosed. The system comprises: a syringe barrel having a needle connector disposed thereon; a hydrogel hydrolysis material disposed in the syringe barrel; and wherein the hydrogel hydrolysis material is configured to be injected into a hydrogel formed in the body of a patient in order to accelerate the hydrolysis of the hydrogel.

Alternatively or additionally to any of the embodiments above, the hydrogel hydrolysis material includes a lipase.

Alternatively or additionally to any of the embodiments above, the hydrogel hydrolysis material includes an esterase.

Alternatively or additionally to any of the embodiments above, the hydrogel hydrolysis material is configured to hydrolyze ester bonds of triglycerides.

Alternatively or additionally to any of the embodiments above, further comprising a complimentary hydrogel-based perirectal spacer system including a needle compatible with the needle connector.

A method for accelerating hydrolysis of a hydrogel-based perirectal spacer is disclosed. The method comprises: delivering the hydrogel-based perirectal spacer into a patient via a delivery syringe system coupled to a needle; disconnecting the delivery syringe system from the needle; attaching a hydrolysis syringe system to the needle, the hydrolysis syringe system including a syringe barrel and a hydrogel hydrolysis material disposed in the syringe barrel; and injecting the hydrogel hydrolysis material into the patient adjacent to the hydrogel-based perirectal spacer in order to accelerate hydrolysis of the hydrogel-based perirectal spacer.

Alternatively or additionally to any of the embodiments above, the hydrogel hydrolysis material includes a lipase.

Alternatively or additionally to any of the embodiments above, the hydrogel hydrolysis material includes an esterase.

Alternatively or additionally to any of the embodiments above, the hydrogel hydrolysis material is configured to hydrolyze ester bonds of triglycerides.

Alternatively or additionally to any of the embodiments above, further comprising observing the delivering the hydrogel-based perirectal spacer into the patient for a complication prior to injecting the hydrogel hydrolysis material into the patient.

Alternatively or additionally to any of the embodiments above, the complication includes misplacement of the hydrogel-based perirectal spacer.

Alternatively or additionally to any of the embodiments above, the complication includes patient discomfort.

Alternatively or additionally to any of the embodiments above, further comprising delivering a replacement hydrogel-based perirectal spacer into the patient.

Alternatively or additionally to any of the embodiments above, further comprising observing the delivering the replacement hydrogel-based perirectal spacer into the patient for a second complication.

Alternatively or additionally to any of the embodiments above, further comprising treating the patient with radiation therapy.

Alternatively or additionally to any of the embodiments above, injecting the hydrogel hydrolysis material into the patient adjacent to the hydrogel-based perirectal spacer includes injecting the hydrogel hydrolysis material into the hydrogel-based perirectal spacer.

A system for accelerating hydrolysis of a hydrogel is disclosed. The system comprises: a syringe barrel having a needle connector disposed thereon, the needle connector being compatible with a needle used with a hydrogel-based perirectal spacer system; wherein the hydrogel-based perirectal spacer system is configured to deliver a hydrogel-based perirectal spacer into a patient; a hydrogel hydrolysis material disposed in the syringe barrel; and wherein the hydrogel hydrolysis material is configured to be injected adjacent to the hydrogel-based perirectal spacer system in order to accelerate the hydrolysis of the hydrogel-based perirectal spacer upon misplacement of the hydrogel-based perirectal spacer.

Alternatively or additionally to any of the embodiments above, the hydrogel hydrolysis material includes a lipase.

Alternatively or additionally to any of the embodiments above, the hydrogel hydrolysis material includes an esterase.

Alternatively or additionally to any of the embodiments above, the hydrogel hydrolysis material is configured to hydrolyze ester bonds of triglycerides.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1 is a schematic overview depicting a region of the human body.

FIG. 2 depicts the placement of a hydrogel-based perirectal spacer in the human body.

FIG. 3 schematically depicts a radiation therapy after placement of a hydrogel-based perirectal spacer in the human body.

FIG. 4 depicts an example system for delivering a hydrogel-based perirectal spacer.

FIG. 5 depicts an example system for hydrogel hydrolysis.

FIG. 6 depicts the delivery of a hydrogel hydrolysis material to a hydrogel-based perirectal spacer.

FIG. 7 depicts the accelerated hydrolysis of a hydrogel-based perirectal spacer.

While the disclosure is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (e.g., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used connection with other embodiments whether or not explicitly described unless clearly stated to the contrary.

The following detailed description should be read with reference to the drawings in which similar elements in different drawings are numbered the same. The drawings, which are not necessarily to scale, depict illustrative embodiments and are not intended to limit the scope of the invention.

In some instances, it may be desirable to place a hydrogel-based perirectal spacer into a patient prior to radiation therapy for prostate cancer. The hydrogel creates a space between the rectum and the prostate, moving the rectum away from the treatment region. This can help to reduce radiation exposure to the rectum and/or provide other desirable benefits. Example hydrogel-based perirectal spacer materials form hydrogels in situ (e.g., in the body of a patient) by mixing a plurality of substances/materials such as a precursor material and an accelerator solution. It can be appreciated that placing the spacer into a patient could lead to complications such as patient pain/discomfort, infiltration of the gel into the rectal wall, urinary retention, etc. Furthermore, misplacement of the hydrogel-based perirectal spacer may reduce the effectiveness of the spacer. Disclosed herein are systems and methods for reducing patient safety risks, improving patient comfort, improving the effectiveness of the spacer, solving spacer misplacement, combinations thereof, and/or other benefits. Such systems may, for example, be used to accelerate the hydrolysis of hydrogels (e.g., such as accelerate the hydrolysis of hydrogel-based perirectal spacers).

In addition, implantable medical devices (e.g., including occlusive hydrogels, occlusive medical devices, embolic materials, embolic medical devices, bulking agents, combinations thereof, and/or the like) are used in a number of different anatomical locations and for a number of different purposes. There may be instances where it may be desirable to hydrolyze and/or disrupt an implantable medical device such as an occlusive hydrogel, an occlusive medical device, an embolic material or device, a bulking agent, combinations thereof, and/or the like. The systems disclosed herein may be used for reducing patient safety risks, improving patient comfort, improving the effectiveness of the spacer, solving spacer misplacement, combinations thereof, and/or other benefits, for example, by accelerating the hydrolysis of implantable medical devices (e.g., including occlusive hydrogels, occlusive medical devices, embolic materials, embolic medical devices, bulking agents, combinations thereof, and/or the like). This may include systems for accelerating the hydrolysis of implantable medical devices at a number of different anatomical locations and for a number of different purposes. In other words, the disclosure is not intended to limit the systems disclosed herein to systems for accelerating the hydrolysis of hydrogel-based perirectal spacers as other systems for accelerating hydrolysis of other medical devices (e.g., including other implantable medical devices such as those disclosed herein) are contemplated.

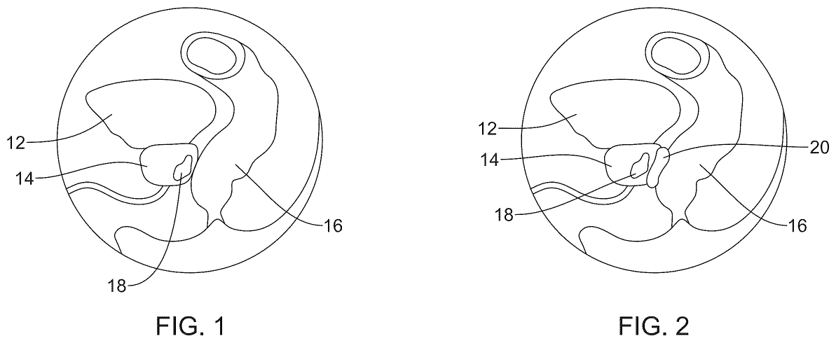

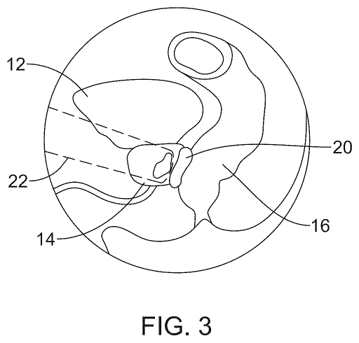

As indicated herein, example systems disclosed herein may be used for accelerating hydrolysis of hydrogel-based perirectal spacers. FIGS. 1-3 illustrate the placement of such example hydrogel-based perirectal spacers. For example, FIG. 1 is a schematic overview depicting a region of the human body, for example including the bladder 12, prostate 14, and rectum 16. In this example, the prostate 14 may include a tumor 18. In some instances, it may be desirable to treat the tumor 18 with radiation therapy. Prior to radiation therapy, it may be desirable to place a hydrogel-based perirectal spacer 20 in the human body as shown in FIG. 2. With the prostate 14 spaced from the rectum 16, radiation therapy (e.g., generally denoted with reference number 22) may be used to treat the tumor 18 as schematically depicted in FIG. 3.

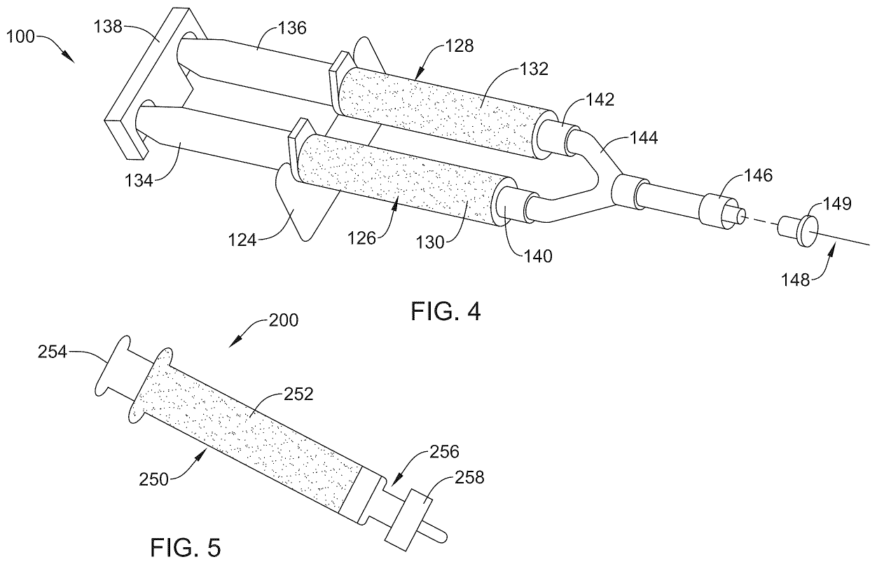

FIG. 4 depicts an example system 100 for delivering a hydrogel-based perirectal spacer. The system 100 may include a syringe holder 124, a first syringe or syringe barrel 126 coupled to the syringe holder 124, and a second syringe or syringe barrel 128 coupled to the syringe holder 124. A first substance or material 130 may be disposed within the first syringe 126. A second substance or material 132 may be disposed within the second syringe 128. The first syringe 126 may include a first plunger 134. The second syringe 128 may include a second plunger 136. A plunger cap 138 may be disposed over the plungers 134, 136. Respective outlets 140, 142 of the syringes 126, 128 may connect with a connector member or Y-connector 144. A needle connector 146 may be disposed at the end of the Y-connector 144. A needle 148 (e.g., a 16-gauge to 23-gauge needle 148; other sizes are contemplated) may be coupled to the needle connector 146. For example, the needle 148 may have a proximal connector 149 that is compatible with the needle connector 146. It can be appreciated that a variety of different systems are contemplated beyond what is shown in FIG. 4.

In use, a clinician may place the needle 148 at the target site. In some instances, the target site may be between the prostate 14 and the rectum 16. The clinician may remove the plunger cap 138 from the plungers 134, 136. The plungers 134, 136 may be depressed to advance the materials 130, 132 within the syringes 126, 128 to the target site, where the materials 130, 132 may form a hydrogel (e.g., may form the hydrogel-based perirectal spacer 20) in situ (e.g., in the body of a patient). With the hydrogel-based perirectal spacer 20, a suitable therapy (e.g., radiation therapy) may be conducted.

FIG. 5 depicts an example system 200 for hydrogel hydrolysis. The system may include a syringe or syringe barrel 250 having a hydrogel hydrolysis material 252 disposed therein. The syringe 250 may include a plunger 254 and an outlet region 256 having a needle connector 258. The form of the system 200 can vary. For example, in some instances, the hydrogel hydrolysis material 252 may be a solution that can be directed injected into hydrogel-based perirectal spacer 20. Alternatively, the hydrogel hydrolysis material 252 may include two or more substances that can be mixed together prior to injection. In such instances, a system similar to the system 100 may be used. Other configurations are contemplated.

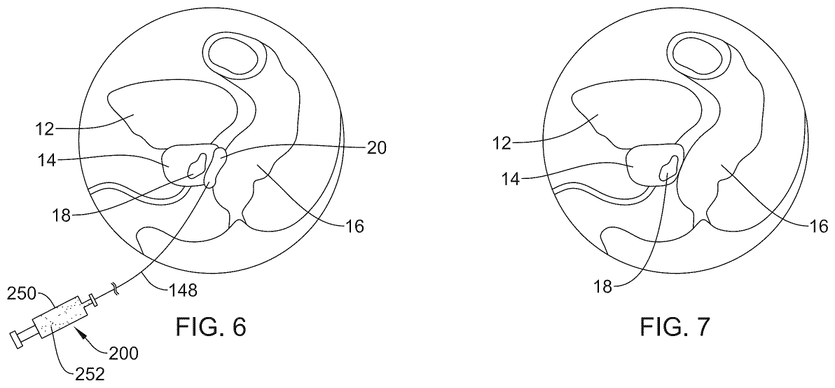

In use, the system 200 can be connected to a needle (e.g., the needle 148 or a different needle), for example by connecting the needle connector 258 to the proximal connector 149 of the needle 148. The plunger 254 may be used to deliver the hydrogel hydrolysis material 252 as schematically depicted in FIG. 6. The hydrogel hydrolysis material 252 may accelerate the hydrolysis of the hydrogel-based perirectal spacer 20 as schematically depicted in FIG. 7.

The hydrogel hydrolysis material 252 may include any one of a variety of materials suitable for accelerating the hydrolysis of a hydrogel. For example, hydrogel-based perirectal spacers such as SpaceOAR™ hydrogels include a poly(ethylene glycol)-based hydrogel with a number of ester bonds. Such ester bonds may have a tendency to hydrolyze slowly over time (e.g., on the order of several months). In order to accelerate the hydrolysis of such materials (e.g., materials with ester bonds), an enzymatic protein capable of hydrolyzing ester bonds (e.g., ester bonds of triglycerides) may be used as the hydrogel hydrolysis material 252. An example of suitable enzymatic protein may include a lipase or an esterase. Example lipases and/or esterases may be non-specific and be capable of hydrolyzing multiple ester bonds (e.g., non-specific binders) or may show selective binding to one specific type of ester bond (e.g., bonding to a specific carbon of a triglyceride). One particular lipase may include a porcine pancreatic lipase. Other enzymatic proteins may include a carboxylesterase, peptidase, amidase, acetylcholinesterase, bile-salt-activated esterase, protein-glutamate methylesterase, cholinesterase, carboxymethylene-butenolidase, crystal protein, cutinase, cAMP-regulated D2 protein, 2-hydroxymuconic semialdehyde hydrolase, gut esterase, esterase B1, liver carboxylesterase, esterase 1, esterase B2, esterase 4, esterase 5, esterase 6, arginine esterase, esterase 5A, esterase 5B, esterase 5C, esterase D, juvenile hormone esterase, esterase P, Pi 6.1 esterase, phosphatidylcholine-sterol acyltransferases, lipase 1, lipase 2, lipase 3, lipase 4, lipase 5, triacylglycerol lipase, lipoprotein lipase, pancreatic lipase, hormone sensitive lipase, lactonizing lipase, mono and diacylglycerol lipase, 6-methylsalicylic acid synthase, phenmedipham hydrolase, poly (3-hydroyalkanoate) depolymerase, 2-hydroxy-6-oxo-2,4-heptadienoate hydrolase, tropinesterase, vitellogenin I, vitellogenin II, vitellogenin III, 2-hydroxymuconic semialdehyde hydrolase, acetyl esterase, protein-glutamate methyl-esterase, S-acyl fatty acid synthase thioesterase, acetyl-hydrolase, erythronolide synthase, gramicidin S biosynthesis GRST protein, triglyceride lipase-cholesterol esterase, Candida Antarctica B lipase, and/or the like. Other materials are contemplated.

The hydrogel hydrolysis material 252 (and/or the system 200, in general) may be provided as a separate system that can be used in conjunction with other systems such as the system 100. In other words, the hydrogel hydrolysis material 252 (and/or the system 200, in general) may be a separate product that is not necessarily tied to another system (e.g., such as the system 100). Alternatively, the system 100 and the system 200 may be provided together as a grouped or singular package. In other words, the hydrogel hydrolysis material 252 (and/or the system 200, in general) and the system 100 may be part of a singular product or package.

When using the hydrogel hydrolysis material 252 (and/or the system 200, in general), a clinician may deliver the hydrogel-based perirectal spacer 20 into a patient using the system 100 (e.g., including the needle 148). In some cases, the clinician may observe the delivery of the hydrogel-based perirectal spacer 20 into the patient for a complication (e.g., such as misplacement of the hydrogel-based perirectal spacer 20, patient discomfort, etc.). If a complication is observed, or for other reasons such as the completion of radiation therapy, it may be desirable to accelerate the hydrolysis of the hydrogel-based perirectal spacer 20). Accordingly, the clinician may disconnect the system 100 from the needle 148 and attach the system 200 to the needle 148 (the system 200 including the hydrogel hydrolysis material 252). The clinician may inject the hydrogel hydrolysis material 252 into the patient adjacent to the hydrogel-based perirectal spacer 20 in order to accelerate hydrolysis of the hydrogel-based perirectal spacer 20. The system 200 can be used right after the clinician observes a complication (e.g., which may be during or right after the procedure to the deliver the hydrogel-based perirectal spacer 20, or hours later, or days later). In some cases, the system 200 can be used with the same needle 148, while the needle 148 is still placed within the patient. In other instances, the system 200 can be used with a different needle that is placed adjacent to the hydrogel-based perirectal spacer 20.

In some cases, it may be desirable to re-deliver a hydrogel-based perirectal spacer 20 after hydrolysis of the first hydrogel-based perirectal spacer 20. For example, if the first hydrogel-based perirectal spacer 20 is misplaced, it may be desirable to hydrolyze the first hydrogel-based perirectal spacer 20 and then replace the first hydrogel-based perirectal spacer 20 with a replacement hydrogel-based perirectal spacer. In such instances, a clinician may deliver a replacement hydrogel-based perirectal spacer 20 into the patient. The process of observing the replacement hydrogel-based perirectal spacer may be similar to that of the first hydrogel-based perirectal spacer 20. For example, the clinician may observe the delivery of the replacement hydrogel-based perirectal spacer 20 into the patient for a second complication and, if desired, accelerate the hydrolysis of the replacement hydrogel-based perirectal spacer 20.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

What is claimed is:

1. A system for hydrogel hydrolysis, the system comprising:
a syringe barrel having a needle connector disposed thereon;
a hydrogel hydrolysis material disposed in the syringe barrel; and
wherein the hydrogel hydrolysis material is configured to be injected into a hydrogel formed in the body of a patient in order to accelerate the hydrolysis of the hydrogel.

2. The system of claim 1, wherein the hydrogel hydrolysis material includes a lipase.

3. The system of claim 1, wherein the hydrogel hydrolysis material includes an esterase.

4. The system of claim 1, wherein the hydrogel hydrolysis material is configured to hydrolyze ester bonds of triglycerides.

5. The system of claim 1, further comprising a complimentary hydrogel-based perirectal spacer system including a needle compatible with the needle connector.

6. A method for accelerating hydrolysis of a hydrogel-based perirectal spacer, the method comprising:
delivering the hydrogel-based perirectal spacer into a patient via a delivery syringe system coupled to a needle;
disconnecting the delivery syringe system from the needle;
attaching a hydrolysis syringe system to the needle, the hydrolysis syringe system including a syringe barrel and a hydrogel hydrolysis material disposed in the syringe barrel; and
injecting the hydrogel hydrolysis material into the patient adjacent to the hydrogel-based perirectal spacer in order to accelerate hydrolysis of the hydrogel-based perirectal spacer.

7. The method of claim 6, wherein the hydrogel hydrolysis material includes a lipase.

8. The method of claim 6, wherein the hydrogel hydrolysis material includes an esterase.

9. The method of claim 6, wherein the hydrogel hydrolysis material is configured to hydrolyze ester bonds of triglycerides.

10. The method of claim 6, further comprising observing the delivering the hydrogel-based perirectal spacer into the patient for a complication prior to injecting the hydrogel hydrolysis material into the patient.

11. The method of claim 10, wherein the complication includes misplacement of the hydrogel-based perirectal spacer.

12. The method of claim 10, wherein the complication includes patient discomfort.

13. The method of claim 6, further comprising delivering a replacement hydrogel-based perirectal spacer into the patient.

14. The method of claim 13, further comprising observing the delivering the replacement hydrogel-based perirectal spacer into the patient for a second complication.

15. The method of claim 6, further comprising treating the patient with radiation therapy.

16. The method of claim 6, wherein injecting the hydrogel hydrolysis material into the patient adjacent to the hydrogel-based perirectal spacer includes injecting the hydrogel hydrolysis material into the hydrogel-based perirectal spacer.

17. A system for accelerating hydrolysis of a hydrogel, the system comprising:
a syringe barrel having a needle connector disposed thereon, the needle connector being compatible with a needle used with a hydrogel-based perirectal spacer system;
wherein the hydrogel-based perirectal spacer system is configured to deliver a hydrogel-based perirectal spacer into a patient;
a hydrogel hydrolysis material disposed in the syringe barrel; and
wherein the hydrogel hydrolysis material is configured to be injected adjacent to the hydrogel-based perirectal spacer system in order to accelerate the hydrolysis of the hydrogel-based perirectal spacer upon misplacement of the hydrogel-based perirectal spacer.

18. The system of claim 17, wherein the hydrogel hydrolysis material includes a lipase.

19. The system of claim 17, wherein the hydrogel hydrolysis material includes an esterase.

20. The system of claim 17, wherein the hydrogel hydrolysis material is configured to hydrolyze ester bonds of triglycerides.
